(19) United States
(12) Patent Application Publication
(10) Pub. No.: US 2019/0298282 A1
(43) Pub. Date: Oct. 3, 2019

(54) RADIATION DETECTOR AND RADIOGRAPHIC IMAGING APPARATUS

(71) Applicant: FUJIFILM Corporation, Tokyo (JP)

(72) Inventors: Munetaka KATO, Kanagawa (JP); Keiichi AKAMATSU, Kanagawa (JP)

(73) Assignee: FUJIFILM Corporation, Tokyo (JP)

(21) Appl. No.: 16/445,221

(22) Filed: Jun. 19, 2019

Related U.S. Application Data
(63) Continuation of application No. PCT/JP2017/046782, filed on Dec. 26, 2017.

(30) Foreign Application Priority Data
Dec. 26, 2016 (JP) 2016-251803
Jun. 28, 2017 (JP) 2017-126683

(51) Int. Cl. A61B 6/00

(52) U.S. Cl. CPC A61B 6/42; A61B 6/54; A61B 6/482

(57) ABSTRACT

Provided are a radiation detector and a radiographic imaging apparatus that can suppress peeling of an end part of a reflective layer. The radiation detector includes a portion in which a TFT substrate; a pixel array that is provided in a pixel region of the TFT substrate and that has a plurality of pixels for accumulating electric charges generated in accordance with light converted from radiation; a conversion layer that converts the radiation into light; a light-transmissive pressure sensitive adhesive layer; a reflective layer that reflects the light converted by the conversion layer; and an adhesive layer that covers at least a region ranging from an end part of the reflective layer to a surface of the substrate are provided in order.

### CROSS-REFERENCE TO RELATED APPLICATIONS

This application is a continuation application of International Application No. PCT/JP2017/046782, filed on Dec. 26, 2017, which is incorporated herein by reference in its entirety. Further, this application claims priority from Japanese Patent Application No. 2016-251803, filed on Dec. 26, 2016, and Japanese Patent Application No. 2017-126683, filed on Jun. 28, 2017, the disclosures of which are incorporated by reference herein.

### BACKGROUND

### Technical Field

The present disclosure relates to a radiation detector and a radiographic imaging apparatus.

### Related Art

In the related art, radiographic imaging apparatuses that perform radiographic imaging for medical diagnosis have been known. A radiation detector for detecting radiation transmitted through a subject and generating a radiographic image is used for such radiographic imaging apparatuses.

As the radiation detector, there is one comprising a pixel array in which a plurality of pixels that accumulates electric charges generated in accordance with light converted from radiation is formed, a conversion layer that converts the radiation into light, a reflective layer that reflects the light converted by the conversion layer, and a protective layer that covers the entire conversion layer and the entire reflective layer (refer to JP2016-128764A and JP2014-185857A).

### SUMMARY

In the radiation detector of the above related art, the protective layer has a function of protecting the conversion layer from moisture, such as humidity, and a function of fixing the reflective layer to a substrate and the conversion layer. However, in the related art, there is a concern that the reflective layer cannot be fixed to the substrate and the conversion layer due to the protective layer. Particularly, there is a case where the fixation of an end part of the reflective layer is not sufficient and peeling is likely to occur.

The present disclosure provides a radiation detector and a radiographic imaging apparatus that can suppress peeling of an end part of a reflective layer.

A radiation detector of a first aspect of the present disclosure comprises a portion in which a substrate; a pixel array that is provided in a pixel region of the substrate and that has a plurality of pixels for accumulating electric charges generated in accordance with light converted from radiation; a conversion layer that converts the radiation into light; a light-transmissive pressure sensitive adhesive layer; a reflective layer that reflects the light converted by the conversion layer; and an adhesive layer that covers at least a region ranging from an end part of the reflective layer to a surface of the substrate are laminated in order.

A radiation detector of a second aspect of the present disclosure comprises a portion in which a substrate; a pixel array that is provided on the substrate via a peeling layer and that has a plurality of pixels for accumulating electric charges generated in accordance with light converted from radiation; a conversion layer that converts the radiation into light; a light-transmissive pressure sensitive adhesive layer; a reflective layer that reflects the light converted by the conversion layer; and an adhesive layer that covers at least a region ranging from an end part of the reflective layer to a surface of the substrate are provided in order.

Additionally, in the radiation detector of a third aspect of the present disclosure based on the first aspect or the second aspect, the conversion layer covers a region of the substrate where the pixel array is provided.

A radiation detector of a fourth aspect of the present disclosure comprises a portion in which a barrier layer; a pixel array that has a plurality of pixels for accumulating electric charges generated in accordance with light converted from radiation; a conversion layer that converts the radiation into light; a light-transmissive pressure sensitive adhesive layer; a reflective layer that reflects the light converted by the conversion layer; and an adhesive layer that covers at least a region ranging from an end part of the reflective layer to a surface of the barrier layer are provided in order.

Additionally, in the radiation detector of a fifth aspect of the present disclosure based on the fourth aspect, the conversion layer covers a region of the barrier layer where the pixel array is provided.

Additionally, in the radiation detector of a sixth aspect of the present disclosure based on any one aspect of the first to fifth aspects, the pressure sensitive adhesive layer covers a region including a central part of the conversion layer.

Additionally, in the radiation detector of a seventh aspect of the present disclosure based on any one aspect of the first to fifth aspects, the pressure sensitive adhesive layer covers the conversion layer in a region enclosing the pixel array.

Additionally, in the radiation detector of an eighth aspect of the present disclosure based on any one aspect of the first to seventh aspects, a peripheral edge part of the conversion layer has an inclination such that a thickness thereof decreases toward an outside, and an outer periphery part of the reflective layer is located at the peripheral edge part of the conversion layer.

Additionally, in the radiation detector of a ninth aspect of the present disclosure based on any one aspect of the first to seventh aspects, a peripheral edge part of the conversion layer has two different heights, and an outer peripheral part of the reflective layer is located at the peripheral edge part of the conversion layer.

Additionally, in the radiation detector of a tenth aspect of the present disclosure based on any one aspect of the first to ninth aspects, the adhesive layer covers the reflective layer.

Additionally, in the radiation detector of an eleventh aspect of the present disclosure based on any one aspect of the first to ninth aspects, the adhesive layer covers the conversion layer.

Additionally, in the radiation detector of a twelfth aspect of the present disclosure based on any one aspect of the first to eleventh aspects, the pressure sensitive adhesive layer is provided between the reflective layer and the conversion layer.

Additionally, in the radiation detector of a thirteenth aspect of the present disclosure based on any one aspect of the first to twelfth aspects, a material of the reflective layer is white PET.

Additionally, in the radiation detector of a fourteenth aspect of the present disclosure based on the thirteenth aspect, a thickness of the reflective layer is 10 μm or more and 40 μm or less.

Additionally, in the radiation detector of a fifteenth aspect of the present disclosure based on any one aspect of the first to fourteenth aspects, the reflective layer is provided in a region corresponding to the pixel array.

Additionally, in the radiation detector of a sixteenth aspect of the present disclosure based on any one aspect of the first to fifteenth aspects, a thickness of the pressure sensitive adhesive layer is 2 μm or more and 7 μm or less.

Additionally, in the radiation detector of a seventeenth aspect of the present disclosure based on any one aspect of the first to sixteenth aspects, the conversion layer includes columnar crystals of CsI.

Additionally, in the radiation detector of an eighteenth aspect of the present disclosure based on any one aspect of the first to sixteenth aspects, the conversion layer is a resin layer in which GOS applied to the pixel array is dispersed.

Additionally, the radiation detector of a nineteenth aspect of the present disclosure based on any one aspect of the first to eighteenth aspects further comprises a protective layer that covers a laminate including the conversion layer, the pressure sensitive adhesive layer laminated on the conversion layer, and the reflective layer laminated on the pressure sensitive adhesive layer, and the adhesive layer.

Additionally, in the radiation detector of a twentieth aspect of the present disclosure based on any one aspect of the first to nineteenth aspects, an inclination angle of at least a portion of the peripheral edge part of the conversion layer is 90° or more and 166° or less.

Additionally, a radiographic imaging apparatus of a twenty first aspect of the present disclosure comprises the radiation detector of the present disclosure; a control unit that outputs control signals for reading electric charges accumulated in the plurality of pixels; a drive unit that allows the electric charges to be read from the plurality of pixels in accordance with the control signals; and a signal processing unit to which electrical signals according to the electric charges read from the plurality of pixels are input, and which generates image data according to the input electrical signals to output the image data to the control unit.

According to the present disclosure, peeling of the end part of the reflective layer can be suppressed.

### BRIEF DESCRIPTION OF THE DRAWINGS

FIG. 1 is a configuration view illustrating an example of a configuration of a TFT substrate in a radiation detector of a first embodiment.

FIG. 2 is a plan view of the example of the radiation detector of the first embodiment as seen from a side on which a conversion layer is provided.

FIG. 3 is a cross-sectional view taken along line A-A of the radiation detector illustrated in FIG. 2.

FIG. 4 is a cross-sectional view for explaining a peripheral edge part and a central part in the conversion layer of the present embodiment.

FIG. 5 is a cross-sectional view illustrating an example of a laminated state of the conversion layer, a pressure sensitive adhesive layer, a reflective layer, an adhesive layer, and a protective layer in the radiation detector of the first embodiment.

FIG. 6 is a view illustrating an example of a method of manufacturing the radiation detector of the first embodiment.

FIG. 7 is a plan view of an example of a radiation detector of a second embodiment as seen from the side on which a conversion layer is provided.

FIG. 8 is a cross-sectional view taken along line A-A of the radiation detector illustrated in FIG. 7.

FIG. 9 is a plan view of an example of a radiation detector of a third embodiment as seen from the side on which a conversion layer is provided.

FIG. 10 is a cross-sectional view taken along line A-A of the radiation detector illustrated in FIG. 9.

FIG. 11 is a plan view of an example of a radiation detector of a fourth embodiment as seen from the side on which a conversion layer is provided.

FIG. 12 is a cross-sectional view taken along line A-A of the radiation detector illustrated in FIG. 11.

FIG. 13 is a plan view of an example of a radiation detector of a fifth embodiment as seen from the side on which a conversion layer is provided.

FIG. 14 is a cross-sectional view taken along line A-A of the radiation detector illustrated in FIG. 13.

FIG. 15 is a cross-sectional view of an example of a radiation detector of a sixth embodiment.

FIG. 16 is a cross-sectional view of another example of the radiation detector of the sixth embodiment.

FIG. 17 is a cross-sectional view of an example of a configuration having two conversion layers that face each other with a pixel array interposed therebetween, in the radiation detector of the sixth embodiment.

FIG. 18 is a cross-sectional view of another example of the configuration having the two conversion layers that face each other with the pixel array interposed therebetween, in the radiation detector of the sixth embodiment.

FIG. 19 is a cross-sectional view illustrating the cross section of an example of a radiation detector in which the size of a pixel region is small, as a modification example of the size of the pixel region.

FIG. 20 is a cross-sectional view illustrating the cross section of an example of a radiation detector in which the size of the pixel region is large, as a modification example of the size of the pixel region.

FIG. 21 is a cross-sectional view illustrating the cross section of an example of a radiation detector in which a region provided with the pressure sensitive adhesive layer is larger than a region provided with the reflective layer, as a modification example regarding the region provided with the reflective layer and the region provided with the pressure sensitive adhesive layer.

FIG. 22 is a cross-sectional view illustrating the cross section of an example of a radiation detector in which the region provided with the reflective layer is larger than the region provided with the pressure sensitive adhesive layer, as a modification example regarding the region provided with the reflective layer and the region provided with the pressure sensitive adhesive layer.

FIG. 23 is a cross-sectional view illustrating the cross section of an example of a radiographic imaging apparatus to which the radiation detector of the embodiment is applied.

FIG. 24 is a cross-sectional view illustrating the cross section of another example of the radiographic imaging apparatus to which the radiation detector of the embodiment is applied.

### DESCRIPTION OF EMBODIMENTS

Hereinafter, embodiments of the invention will be described in detail with reference to the drawings. In addition, the present embodiments do not limit the invention.

### First Embodiment

A radiation detector of the present embodiment has a function of detecting radiation transmitted through a subject to output image information representing a radiographic image of the subject. The radiation detector of the present embodiment comprises a thin film transistor (TFT) substrate, and a conversion layer that converts radiation into light (refer to a TFT substrate 12 and a conversion layer 14 of a radiation detector 10 in FIG. 3).

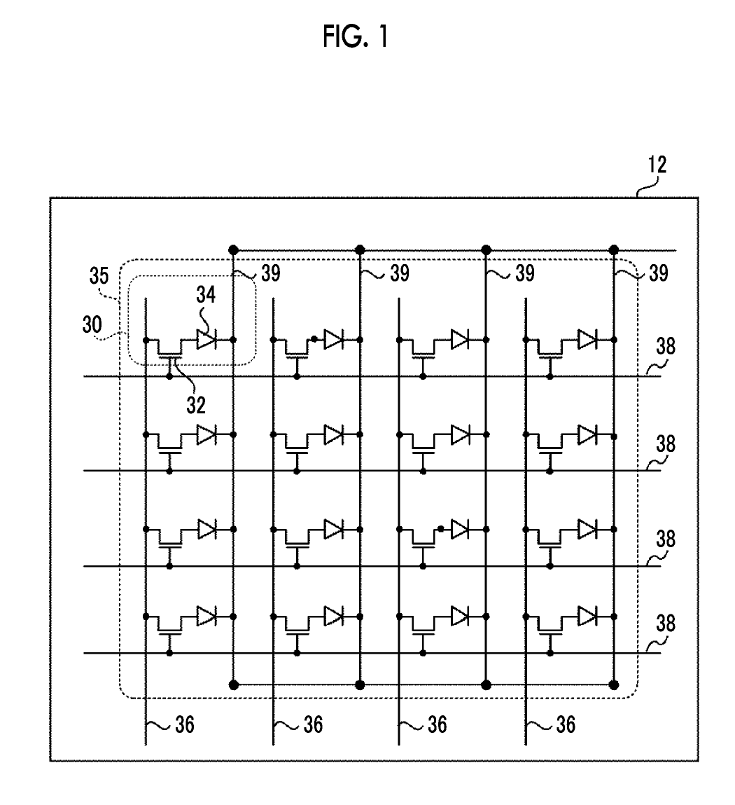

First, an example of the configuration of the TFT substrate 12 in the radiation detector of the present embodiment will be described with reference to FIG. 1. In addition, the TFT substrate 12 of the present embodiment is a substrate in which a pixel array 31 including a plurality of pixels 30 is formed in a pixel region 35 of a base material 11. Therefore, in the following description, the expression “the pixel region 35” is used as the same meaning as “the pixel array 31”. The TFT substrate 12 of the present embodiment is an example of a substrate of the disclosed technique.

The base material 11 is, for example, a glass substrate, such as alkali-free glass, a resin sheet including plastic, such as polyimide, or the like. A specific example of the resin sheet is XENOMAX (registered trademark). Additionally, the base material 11 may have flexibility. In this case, the above resin sheet, a relatively thin glass substrate, or the like is preferable as the base material 11. Taking flexibility into consideration, for example, in a case where the base material 11 is the resin sheet, it is preferable that the thickness thereof is 5 μm to 125 μm. Additionally, for example, in a case where the base material 11 is the glass substrate, generally, the base material 11 has flexibility in a case where the thickness thereof is 0.3 mm or less in a size in which one side is 43 cm or less. Therefore, it is preferable that the thickness is 0.3 mm or less.

Each of the pixels 30 includes a sensor unit 34 and a switching element 32. The sensor unit 34 generates and accumulates an electric charge in accordance with the light converted by the conversion layer. The switching element 32 reads the electric charge accumulated in the sensor unit 34. In the present embodiment, as an example, a thin film transistor (TFT) is used as the switching element 32. For that reason, in the following description, the switching element 32 is referred to as a “TFT 32”.

The plurality of pixels 30 is two-dimensionally disposed in one direction (a scanning wiring direction corresponding to a transverse direction of FIG. 1, hereinafter referred to as a “row direction”), and a direction (a signal wiring direction corresponding to a longitudinal direction of FIG. 1, hereinafter referred as a “column direction”) intersecting the row direction in the pixel region 35 of the TFT substrate 12. Although an array of the pixels 30 is illustrated in a simplified manner in FIG. 1, for example, 1024×1024 pixels 30 are disposed in the row direction and the column direction.

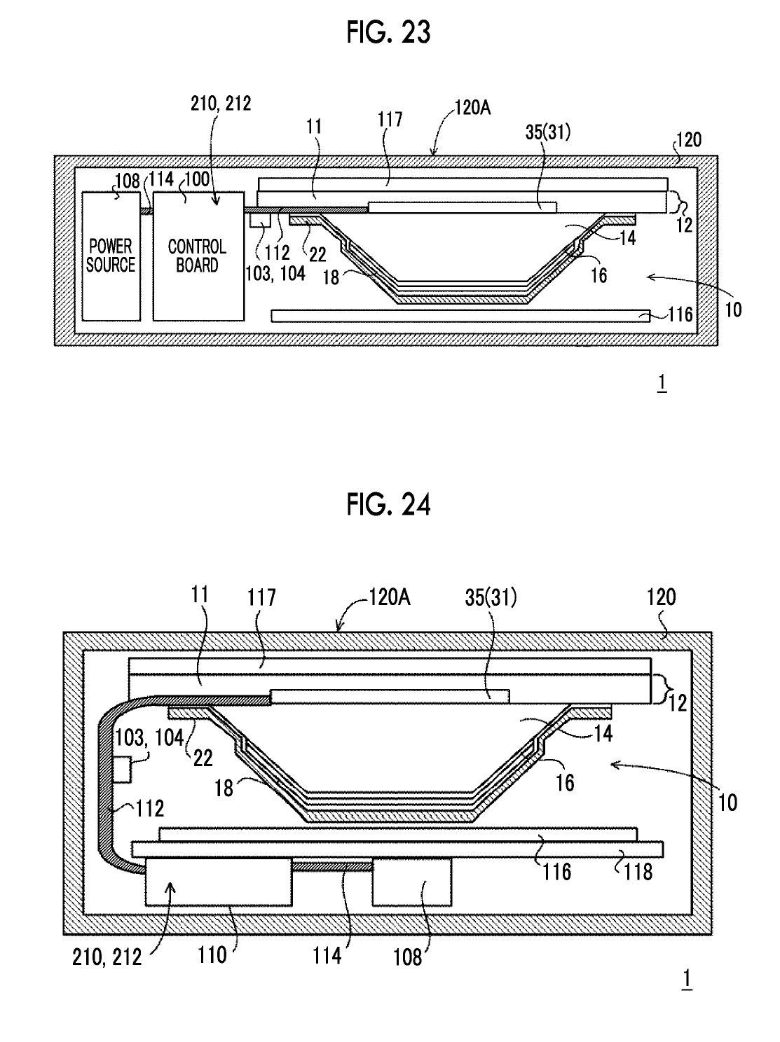

Additionally, a plurality of scanning wiring lines 38 for controlling switching states (ON and OFF) of the TFTs 32, and a plurality of signal wiring lines 36, which is provided for respective columns of the pixels 30 and from which electric charges accumulated in the sensor units 34 are read, are provided in a mutually intersecting manner in the radiation detector 10. The plurality of scanning wiring lines 38 is respectively connected to a drive unit (refer to a drive unit 103 in FIGS. 23 and 24) outside the radiation detector 10 via pads (not illustrated), respectively, provided in the TFT substrate 12, and thereby, control signals, which are output from the drive unit to control the switching states of the TFTs 32, flow to the plurality of scanning wiring lines 38, respectively. Additionally, the plurality of signal wiring lines 36 is respectively connected to a signal processing unit (refer to a signal processing unit 104 in FIGS. 23 and 24) outside the radiation detector 10 via pads (not illustrated), respectively, provided in the TFT substrate 12, and thereby, electric charges read from the respective pixels 30 is output to the signal processing unit.

Additionally, common wiring lines 39 are provided in a wiring direction of the signal wiring lines 36 at the sensor units 34 of the respective pixels 30 in order to apply bias voltages to the respective pixels 30. Bias voltages are applied to the respective pixels 30 from a bias power source by connecting the common wiring lines 39 to the bias power source outside the radiation detector 10 via pads (not illustrated) provided in the TFT substrate 12.

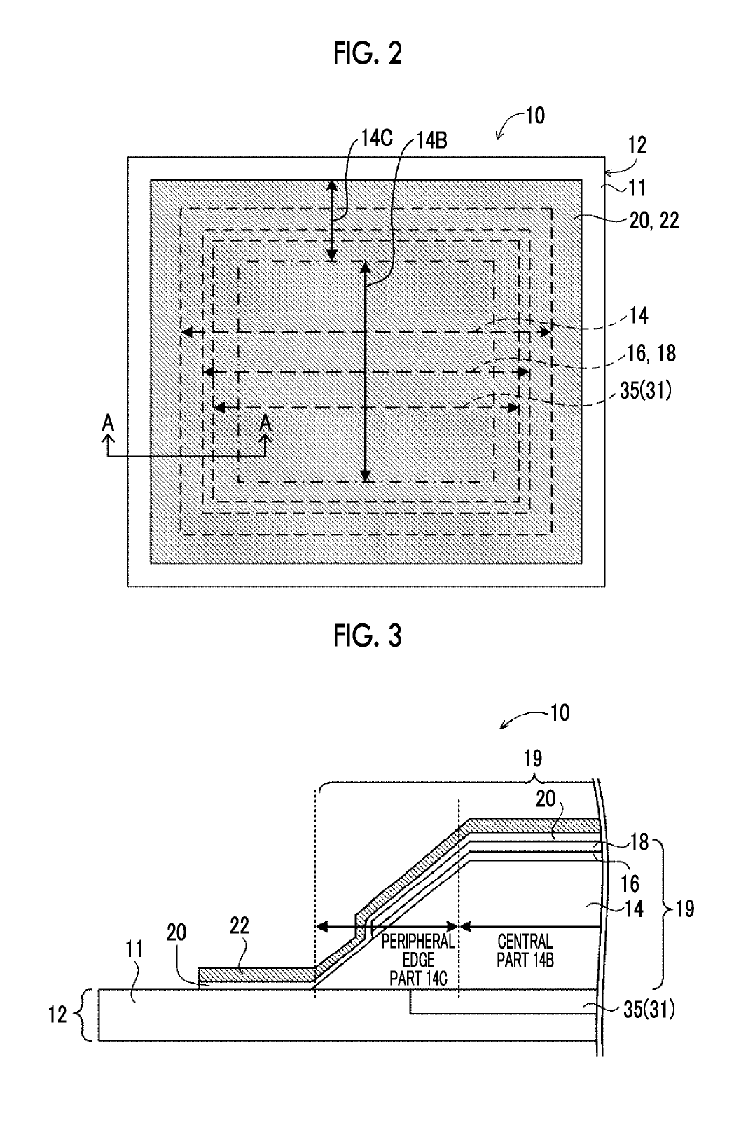

In the radiation detector 10 of the present embodiment, the conversion layer 14 is formed on the TFT substrate 12. FIG. 2 is a plan view of the radiation detector 10 of the present embodiment as seen from a side on which the conversion layer 14 is formed. Additionally, FIG. 3 is a cross-sectional view taken along line A-A of the radiation detector 10 in FIG. 2. In addition, in the following description, the term “on” in the structure of the radiation detector 10 means “on” in a positional relationship with reference to the TFT substrate 12 side.

As illustrated in FIGS. 2 and 3, the conversion layer 14 of the present embodiment is provided on a partial region including the pixel region 35 of the TFT substrate 12. In this way, the conversion layer 14 of the present embodiment is not provided on the region of an outer peripheral part of the TFT substrate 12.

In the present embodiment, a scintillator including CsI (cesium iodide) is used as an example of the conversion layer 14. It is preferable that such a scintillator includes, for example, CsI:Tl (cesium iodide to which thallium is added) or CsI:Na (cesium iodide to which sodium is added) having an emission spectrum of 400 nm to 700 nm at the time of X-ray radiation. In addition, the emission peak wavelength in a visible light region of CsI:Tl is 565 nm.

In the radiation detector 10 of the present embodiment, as in an example illustrated in FIG. 5, the conversion layer 14 is directly formed on the TFT substrate 12 as strip-shaped columnar crystals 14A by vapor-phase deposition methods, such as a vacuum vapor deposition method, a sputtering method, and a chemical vapor deposition (CVD) method. For example, in a case where CsI:Tl is used as the conversion layer 14, a vacuum vapor deposition method is used as a method of forming the conversion layer 14. In the vacuum vapor deposition method, CsI:Tl is heated and gasified by heating means, such as a resistance heating-type crucible in an environment with the vacuum degree of 0.01 Pa to 10 Pa, and CsI:Tl is deposited on the TFT substrate 12 with the temperature of the TFT substrate 12 as the room temperature (20° C.) to 300° C. As the thickness of the conversion layer 14, 100 μm to 800 μm is preferable.

In addition, in the present embodiment, end parts of columnar crystals 14A of the conversion layer 14 on a base point side (a TFT substrate 12 side in the present embodiment) in a growth direction is referred to as “roots”, and sharpened end parts opposite to the roots in the growth direction are referred to as “tips”.

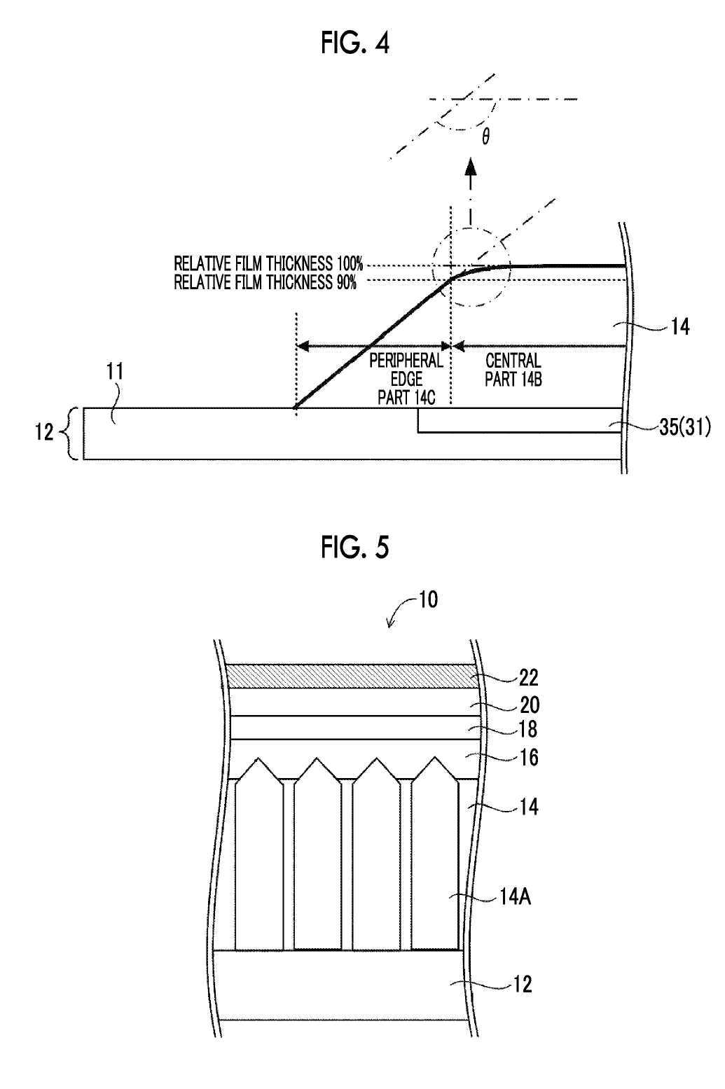

Additionally, since the conversion layer 14 of the present embodiment is formed by the vapor-phase deposition methods as described above, as illustrated in FIG. 3, the thickness of the outer peripheral part of the conversion layer 14 tends to decrease toward the outside as viewed as a whole. Therefore, the conversion layer 14 has an inclination with an inclination angle θ such that the thickness thereof decreases toward the outside. In the present embodiment, an average value of thicknesses of the conversion layer 14, which are regarded as being substantially constant in a case where a manufacturing error and a measurement error is neglected and are within a predetermined range from the center of the conversion layer 14, is adopted as a reference, and as illustrated in FIG. 4, as an example, an outer peripheral region where a relative film thickness (hereinafter referred to as “relative film thickness”) to a reference thickness is 90% or less is referred to as a “peripheral edge part (peripheral edge part 14C)”. Additionally, as illustrated in FIG. 4, the region of the conversion layer 14 surrounded by the peripheral edge part 14C is referred to as a “central part (central part 14B)”. In other words, the “central part” means a region that includes at least a portion in which the thickness of the conversion layer 14 is substantially constant and that also includes a portion in which the relative film thickness exceeds 90%. In the present embodiment, as a specific example, an outer peripheral region, which is within a region of less than 5 mm from the outer periphery of the conversion layer 14 and has a relative film thickness of 90% or less, is referred to as a “peripheral edge part (peripheral edge part 14C)”. For that reason, as illustrated in FIG. 3, FIG. 4, and the like, in the peripheral edge part 14C, the thickness of the conversion layer 14 tends to gradually decrease toward the outer periphery. In other words, the peripheral edge part 14C has two different heights.

Additionally, as illustrated in FIG. 4, the inclination angle θ of an end part of the conversion layer 14 tends to be steep, that is, smaller. Generally, the radiation detector 10 is used in a state where the radiation detector 10 is housed in a housing (refer to a housing 120 in FIG. 23 and FIG. 24). Generally, in many cases, the housing 120 is a so-called narrow frame having a short distance from a side surface of the housing 120 to the end part of the radiation detector 10. For that reason, as described above, the inclination angle θ of the end part of the conversion layer 14 is steep, and is 90° or more and 166° or less as a specific example. In addition, the inclination angle θ of the present embodiment is an example of the inclination angle of the present disclosure.

In addition, the method of measuring the inclination angle θ is not particularly limited. However, in the present embodiment, as an example, in the method of measuring the inclination angle θ, portions of an end part of the conversion layer 14 were peeled from the TFT substrate 12 at the positions of four spots with regular intervals at one side of a rectangular conversion layer 14 and were obtained as respective samples. Measurement was performed by observing the four samples using an optical microscope after the four samples were polished and sectioned. An average value of measurement values of the four samples was set as the inclination angle θ at the side of the conversion layer 14 where the samples were prepared.

Moreover, as illustrated in FIGS. 2 to 5, the radiation detector 10 of the present embodiment comprises a pressure sensitive adhesive layer 16, a reflective layer 18, an adhesive layer 20, and a protective layer 22.

As illustrated in FIGS. 2 and 3 as an example, the pressure sensitive adhesive layer 16 is provided on a region including a portion of the peripheral edge part 14C and the entire central part 14B of the conversion layer 14. In other words, the pressure sensitive adhesive layer 16 of the present embodiment is provided between the conversion layer 14 and the reflective layer 18. In addition, the pressure sensitive adhesive layer 16 is provided at least at a position where the pressure sensitive adhesive layer 16 covers a region where the conversion layer 14 covers the pixel array 31. In other words, the pressure sensitive adhesive layer 16 is provided at a position where the pressure sensitive adhesive layer 16 covers a region where the conversion layer 14 encloses the pixel array 31. Additionally, as illustrated in FIG. 5, in the radiation detector 10 of the present embodiment, the tip of the conversion layer 14 penetrates into the pressure sensitive adhesive layer 16.

The pressure sensitive adhesive layer 16 of the present embodiment is a light-transmissive layer, and examples of the material of the pressure sensitive adhesive layer 16 include an acrylic pressure sensitive adhesive, a hot-melt pressure sensitive adhesive, a silicone adhesive, and the like. Examples of the acrylic pressure sensitive adhesive include urethane acrylate, acrylic resin acrylate, epoxy acrylate, and the like. Examples of the hot-melt pressure sensitive adhesive include thermoplastics, such as ethylene-vinyl acetate copolymer resin (EVA), ethylene-acrylate copolymer resin (EAA), ethylene-ethyl acrylate copolymer resin (EEA), and ethylene-methyl methacrylate copolymer (EMMA).

As the thickness of the pressure sensitive adhesive layer 16 increases (that is, as the spacing between the conversion layer 14 and the reflective layer 18 increases), the light converted by the conversion layer 14 is blurred within the pressure sensitive adhesive layer 16. Therefore, the radiographic image obtained by the radiation detector 10 becomes a blurred image as a result. For that reason, as the thickness of the pressure sensitive adhesive layer 16 increases, a modulation transfer function (MTF) and detective quantum efficiency (DQE) decreases, and the degree of the decrease also increases.

On the other hand, in a case where the thickness of the pressure sensitive adhesive layer 16 is made too small also including a case where the pressure sensitive adhesive layer 16 is not provided, there is a case where a minute air space (not illustrated) is formed between the conversion layer 14 and the reflective layer 18. In this case, multiple reflection of the light from the conversion layer 14 toward the reflective layer 18 occurs between the air space and the conversion layer 14 and between the air space and the reflective layer 18. In a case where light is attenuated due to the multiple reflections, the sensitivity of the radiation detector 10 decreases.

As a result of studies by the present inventors, in a case where the thickness of the pressure sensitive adhesive layer 16 exceeds 7 μm, it turned out that the degree of DQE decrease becomes much larger, and becomes lower than that in a case where the pressure sensitive adhesive layer 16 is not provided (in a case where thickness is 0 μm). Additionally, in a case where the thickness of the pressure sensitive adhesive layer 16 is less than 2 μm, it turned out that the sensitivity of the radiation detector 10 decreases.

Thus, in the present embodiment, the thickness of the pressure sensitive adhesive layer 16 is set to 2 μm or more and 7 μm or less. In addition, although also varying depending on materials, the refractive index of the pressure sensitive adhesive layer 16 is approximately 1.5.

In addition, the pressure sensitive adhesive layer 16 has a function of fixing the reflective layer 18 to the conversion layer 14. However, in a case where the thickness of the pressure sensitive adhesive layer 16 is 2 μm or more, it is possible to obtain a sufficient effect of suppressing that the reflective layer 18 shifts in an in-plane direction (a direction intersecting a thickness direction) with respect to the conversion layer 14.

Meanwhile, as illustrated in FIGS. 2 and 3 as an example, the reflective layer 18 is provided on the pressure sensitive adhesive layer 16 and covers the entire upper surface of the pressure sensitive adhesive layer 16 itself. Additionally, the reflective layer 18 covers at least the central part 14B of the conversion layer 14, and an outer peripheral part of the reflective layer 18 reaches the peripheral edge part 14C of the conversion layer 14. That is, the outer peripheral part of the reflective layer 18 is located at the peripheral edge part 14C of the conversion layer 14 where the thickness decreases toward the outside. In other words, the peripheral edge part 14C of the conversion layer 14 has two different heights, and the outer peripheral part of the reflective layer 18 is located at the peripheral edge part 14C of the conversion layer 14. The reflective layer 18 has a function of reflecting the light converted by the conversion layer 14.

As a material of the reflective layer 18, it is preferable to use an organic material, and it is preferable to use, for example, at least one of white polyethylene terephthalate (PET), TiO₂, Al₂O₃, foamed white PET, a polyester-based high-reflection sheet, specular reflection aluminum, or the like. Particularly, it is preferable to use the white PET as the material from a viewpoint of reflectivity.

In addition, the white PET is obtained by adding a white pigment, such as TiO₂ or barium sulfate, to PET. Additionally, the polyester-based high-reflection sheet is a sheet (film) having a multilayer structure in which a plurality of thin polyester sheets is laminated. Additionally, the foamed white PET is white PET of which the surface is porous.

In the present embodiment, the thickness of the reflective layer 18 is 10 μm or more and 40 μm or less. In a case where the thickness of the reflective layer 18 increases, a level difference between an upper surface of the outer peripheral part of the reflective layer 18 and an upper surface of the conversion layer 14 increases. In the present embodiment, the radiation detector 10 is manufactured by bonding sheets (films) of the adhesive layer 20 and the protective layer 22 to the TFT substrate 12 in a state where layers up to the reflective layer 18 are formed. In a case where the above level difference is large and in a case where the adhesive layer 20 and the protective layer 22 are bonded onto the reflective layer 18, there is a case where at least one of the adhesive layer 20 or the protective layer 22 comes off in the level-difference portion.

Additionally, in a case where the thickness of the reflective layer 18 increases, there is so-called stiffness. Therefore, there is a case where the reflective layer 18 is not easily bent along the inclination of the peripheral edge part 14C of the conversion layer 14 and is not easily processed.

As a result of having studied from these viewpoints, in the radiation detector 10 of the present embodiment, in a case where the white PET is used as a material of the reflective layer 18, the thickness of the reflective layer 18 is 40 μm or less as described above.

On the other hand, as the thickness of the reflective layer 18 decreases, reflectivity decreases. In a case where the reflectivity decreases, the image quality of a radiographic image to be obtained by the radiation detector 10 also tends to deteriorate. For that reason, it is preferable to determine the lower limit of the thickness of the reflective layer 18 in consideration of a desired reflectivity (for example, 80%) from a viewpoint of the image quality of a radiographic image to be obtained by the radiation detector 10. In the radiation detector 10 of the present embodiment, in a case where the white PET is used as a material of the reflective layer 18, the thickness of the reflective layer 18 is 10 μm or more as described above.

Meanwhile, as illustrated in FIGS. 2 and 3 as an example, the adhesive layer 20 is provided from a region in the vicinity of the outer peripheral part of the conversion layer 14 in the TFT substrate 12 to a region covering the end part of the reflective layer 18. In other words, in the radiation detector 10 of the present embodiment, the adhesive layer 20 that covers the entire conversion layer 14 in which the pressure sensitive adhesive layer 16 and the reflective layer 18 are provided is directly fixed (adhered) to a portion of the surface of the TFT substrate 12. The adhesive layer 20 has a function of fixing the reflective layer 18 to the TFT substrate 12 and the conversion layer 14. Additionally, the adhesive layer 20 has a function of fixing the protective layer 22. Examples of the material of the adhesive layer 20 include the same materials as the pressure sensitive adhesive layer 16. In addition, in the present embodiment, the adhesive force of the adhesive layer 20 is stronger than the adhesive force of the pressure sensitive adhesive layer 16.

Moreover, as illustrated in FIGS. 2 and 3 as an example, the protective layer 22 is provided on the adhesive layer 20. The protective layer 22 of the present embodiment covers a laminate 19 and the adhesive layer 20. The laminate 19 includes the conversion layer 14, the pressure sensitive adhesive layer 16 laminated on the conversion layer 14, and the reflective layer 18 laminated on the pressure sensitive adhesive layer 16. The protective layer 22 of the present embodiment has a function of protecting the conversion layer 14 from moisture, such as humidity. Additionally, the protective layer 22 of the present embodiment has a function of fixing the reflective layer 18 to the TFT substrate 12 and the conversion layer 14 together with the adhesive layer 20. The material of the protective layer 22 includes, for example, an organic film. Examples of the organic film include PET, polyphenylene sulfide (PPS), oriented polypropylene (OPP), polyethylene naphthalate (PEN), polyimide (PI), and the like. Additionally, as the protective layer 22, an ALPET (registered trademark) sheet obtained by laminating aluminum, for example by adhering aluminum foil, to an insulating sheet (film), such as polyethylene terephthalate may be used.

An example of a method of manufacturing the radiation detector 10 of the present embodiment illustrated in FIGS. 2 to 5 includes the following method.

A sheet (film) of the pressure sensitive adhesive layer 16 having a desired size tailored for the radiation detector 10 and a sheet of the reflective layer 18 having a desired size are bonded together and prepared in advance.

Meanwhile, the conversion layer 14 is formed directly on the TFT substrate 12, by the vapor-phase deposition methods as described above.

Moreover, the adhesive layer 20 is provided on one entire surface of the sheet of the protective layer 22 having the desired size. In addition, the method of providing the adhesive layer 20 may be a method according to the material of the adhesive layer 20. For example, the sheet of the adhesive layer 20 having the desired size may be bonded to one surface of the protective layer 22, or a liquid material to be the adhesive layer 20 may be applied to the one entire surface of the protective layer 22.

Thereafter, the sheet of the protective layer 22 on which the adhesive layer 20 is provided is bonded to the sheets in a state where the reflective layer 18 and the pressure sensitive adhesive layer 16 are laminated, and a laminated film in which the pressure sensitive adhesive layer 16, the reflective layer 18, the adhesive layer 20, and the protective layer 22 are laminated in this order is obtained.

Moreover, then, the above laminated film is bonded to the TFT substrate 12 on which the conversion layer 14 is formed, in a state where the entire conversion layer 14 is covered.

In addition, in a case where the above bonding is performed, the bonding is performed under the atmospheric pressure or under reduced pressure (under vacuum). However, in order to suppress entry of air or the like while being bonded to each other, it is preferable to perform the bonding under reduced pressure.

Additionally, as a method of providing the reflective layer 18, a method of bonding the sheet-like reflective layer 18 as described above is preferable. A different method includes, for example, a method of forming the reflective layer 18 by applying a binder serving as a material onto the conversion layer 14. However, in the method of applying the binder, the binder applied onto the peripheral edge part 14C of the conversion layer 14 flows along the inclination of the peripheral edge part 14C. Therefore, there is a case where the reflective layer 18 can be formed only at the central part 14B of the conversion layer 14. In this case, in a case where the size of a region where the reflective layer 18 is provided is made small in accordance with the central part 14B of the conversion layer 14, the sensitivity of the radiation detector 10 decreases. Meanwhile, in order to maintain the size of the region where the reflective layer 18 is provided, the size of the radiation detector 10 is increased in a case where the entire conversion layer 14 is enlarged. For that reason, in a case where the conversion layer 14 of the radiation detector 10 has the inclination as in the present embodiment, it is preferable to provide the reflective layer 18 by bonding the sheet-like reflective layer 18.

In this way, in the radiation detector 10 of the present embodiment illustrated in FIGS. 2 to 5, the adhesive layer 20 and the protective layer 22 cover the entire (the entire upper surface and the side surface) reflective layer 18. Additionally, the adhesive layer 20 and the protective layer 22 are directly fixed onto the TFT substrate 12.

Accordingly, according to the radiation detector 10 of the present embodiment, the peeling of the reflective layer 18, particularly the peeling of the end part of the reflective layer 18 is suppressed. In addition, in the present embodiment, the end part of the reflective layer 18 means a region within a predetermined range in the vicinity of the outer periphery of the reflective layer 18.

Additionally, as described above, in a case where the inclination angle is steep at 90° or more and 166° or less at an end part of at least a portion of the conversion layer 14, the reflective layer 18 is easily peeled at an inclined part of the conversion layer 14 compared to a case where the inclination angle θ is gentle.

In contrast, in the radiation detector 10 of the present embodiment, by virtue of the above configuration, the peeling of the reflective layer 18, particularly the peeling of the reflective layer 18 at the inclined part of the conversion layer 14 is suppressed.

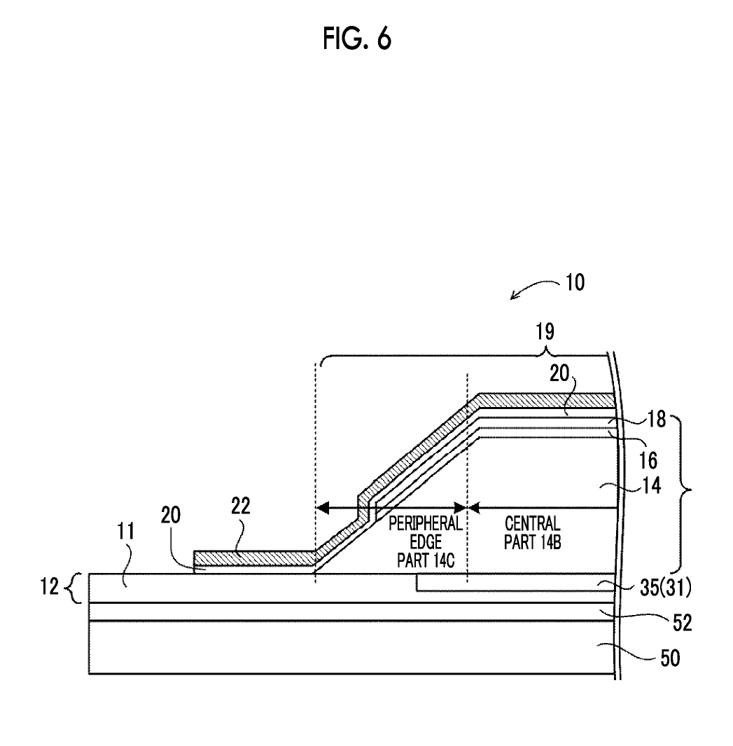

In addition, in the radiation detector 10 of the present embodiment illustrated in FIGS. 2 to 5, in the case of a glass substrate in which the thickness of the base material 11 is relatively small, the conversion layer 14, the pressure sensitive adhesive layer 16, the reflective layer 18, the adhesive layer 20, and the protective layer 22 may be sequentially formed on the TFT substrate 12 as described above. Meanwhile, in a case where the base material 11 is a relatively thin substrate, for example, a substrate having flexibility, as in an example illustrated in FIG. 6, the TFT substrate 12 is formed on a support 50, such as a glass substrate having a thickness larger than the base material 11, via a peeling layer 52, for example by a lamination method or the like. Moreover, similarly to the above, the TFT substrate 12 is peeled from the support 50 by the peeling layer 52 after the conversion layer 14, the pressure sensitive adhesive layer 16, the reflective layer 18, the adhesive layer 20, and the protective layer 22 are sequentially formed. The peeling method is not particularly limited. For example, in a lamination method, mechanical peeling may be performed by using any of the four sides of the TFT substrate 12 (base material 11) as a starting point for peeling and gradually peeling the TFT substrate 12 off from the support 50 toward an opposite side from the side to be the starting point. Additionally, for example, in a laser peeling (laser lift-off) method, the TFT substrate 12 may be peeled from the support 50 by radiating a laser beam from a back surface (for a surface opposite to the surface on which the TFT substrate 12 is provided) of the support 50 and by decomposing the peeling layer 52 with the laser beam transmitted through the support 50.

### Second Embodiment

Next, a second embodiment will be described. In addition, since the radiation detector 10 of the present embodiment is different from the first embodiment in terms of the regions where the pressure sensitive adhesive layer 16 and the reflective layer 18 are provided, the region where the pressure sensitive adhesive layer 16 and the reflective layer 18 are provided will be described with reference to the drawings.

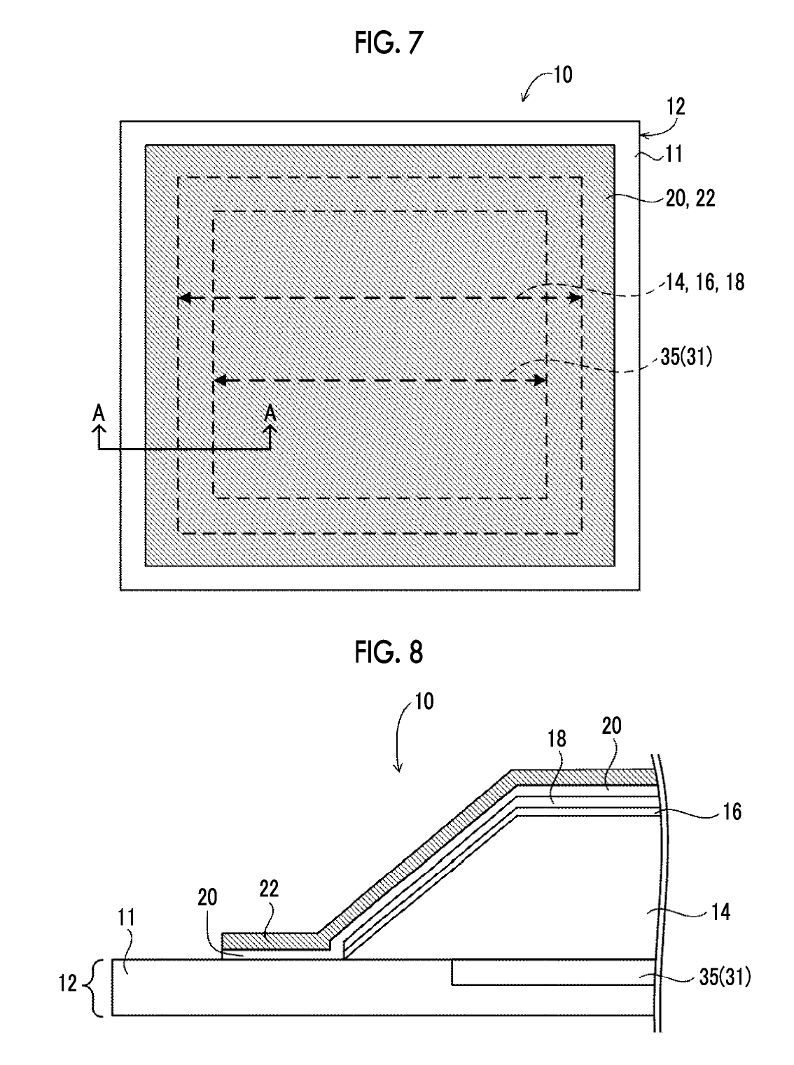

FIG. 7 is a plan view of the radiation detector 10 of the present embodiment as seen from the side on which the conversion layer 14 is formed. Additionally, FIG. 8 is a cross-sectional view taken along line A-A of the radiation detector 10 in FIG. 7.

As illustrated in FIGS. 7 and 8, in the radiation detector 10 of the present embodiment, the pressure sensitive adhesive layer 16 and the reflective layer 18 are provided in the entire region on the conversion layer 14 including the central part and the peripheral edge part. In other words, the pressure sensitive adhesive layer 16 and the reflective layer 18 of the present embodiment cover the entire upper surface of the conversion layer 14. Meanwhile, the pressure sensitive adhesive layer 16 and the reflective layer 18 of the present embodiment are not directly provided on the TFT substrate 12.

In addition, the radiation detector 10 of the present embodiment can be manufactured, for example, by the same manufacturing method as the manufacturing method of the radiation detector 10 that has been described above in the first embodiment.

In this way, also in the radiation detector 10 of the present embodiment illustrated in FIGS. 7 and 8, the adhesive layer 20 and the protective layer 22 cover the entire (the entire upper surface and the side surface) reflective layer 18, similarly to the radiation detector 10 of the first embodiment. Additionally, the adhesive layer 20 and the protective layer 22 are directly fixed onto the TFT substrate 12.

Accordingly, according to the radiation detector 10 of the present embodiment, the peeling of the reflective layer 18, particularly the peeling of the end part of the reflective layer 18 is suppressed.

Additionally, according to the radiation detector 10 of the present embodiment, the reflective layer 18 is larger compared to the radiation detector 10 of the first embodiment, and the entire upper surface of the conversion layer 14 is covered. Therefore, the light from the conversion layer 14 is easily reflected.

### Third Embodiment

Next, a third embodiment will be described. In addition, since the radiation detector 10 of the present embodiment is different from the respective embodiments in terms of the regions where the pressure sensitive adhesive layer 16 and the reflective layer 18 are provided, the regions where the pressure sensitive adhesive layer 16 and the reflective layer 18 are provided will be described with reference to the drawings.

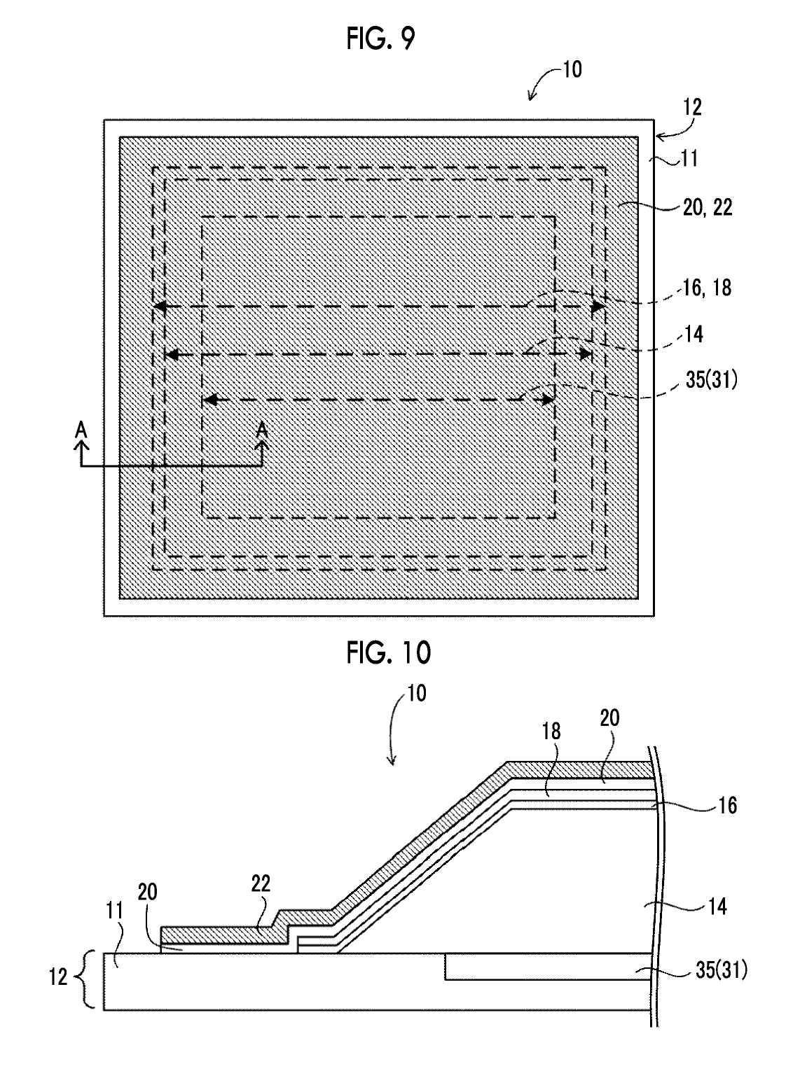

FIG. 9 is a plan view of the radiation detector 10 of the present embodiment as seen from the side on which the conversion layer 14 is formed. Additionally, FIG. 10 is a cross-sectional view taken along line A-A of the radiation detector 10 in FIG. 9.

As illustrated in FIGS. 9 and 10, similarly to the radiation detector 10 of the second embodiment, in the radiation detector 10 of the present embodiment, the pressure sensitive adhesive layer 16 and the reflective layer 18 are provided in the entire region on the conversion layer 14 including the central part and the peripheral edge part. In other words, the pressure sensitive adhesive layer 16 and the reflective layer 18 of the present embodiment cover the entire upper surface of the conversion layer 14. Unlike the radiation detector 10 of the second embodiment, in the radiation detector 10 of the present embodiment, the regions where the pressure sensitive adhesive layer 16 and the reflective layer 18 are provided reach the TFT substrate 12 in the vicinity of the outer periphery of the conversion layer 14. In other words, in the radiation detector 10 of the present embodiment, the pressure sensitive adhesive layer 16 and the reflective layer 18 are directly provided on the TFT substrate 12 in the vicinity of the outer periphery of the conversion layer 14.

In addition, the radiation detector 10 of the present embodiment can be manufactured, for example, by the same manufacturing method as the manufacturing method of the radiation detector 10 that has been described above in the first embodiment.

In this way, also in the radiation detector 10 of the present embodiment illustrated in FIGS. 9 and 10, the adhesive layer 20 and the protective layer 22 cover the entire (the entire upper surface and the side surface) reflective layer 18, similarly to the radiation detector 10 of the first embodiment. Additionally, the adhesive layer 20 and the protective layer 22 are directly fixed onto the TFT substrate 12.

Accordingly, according to the radiation detector 10 of the present embodiment, the peeling of the reflective layer 18, particularly the peeling of the end part of the reflective layer 18 is suppressed.

Additionally, in the radiation detector 10 of the present embodiment, the pressure sensitive adhesive layer 16 and the reflective layer 18 reach the TFT substrate 12. Therefore, although there is a case where the size of the TFT substrate 12 is increased correspondingly, the reflective layer 18 can be more stably fixed.

Moreover, in the radiation detector 10 of the present embodiment, the reflective layer 18 is larger compared to the radiation detector 10 of the first embodiment and the entire upper surface of the conversion layer 14 is covered. Therefore, the light from the conversion layer 14 is easily reflected.

### Fourth Embodiment

Next, a fourth embodiment will be described. In addition, since the radiation detector 10 of the present embodiment is different from the first embodiment in terms of a region where the adhesive layer 20 is provided, the region where the adhesive layer 20 is provided will be described with reference to the drawings.

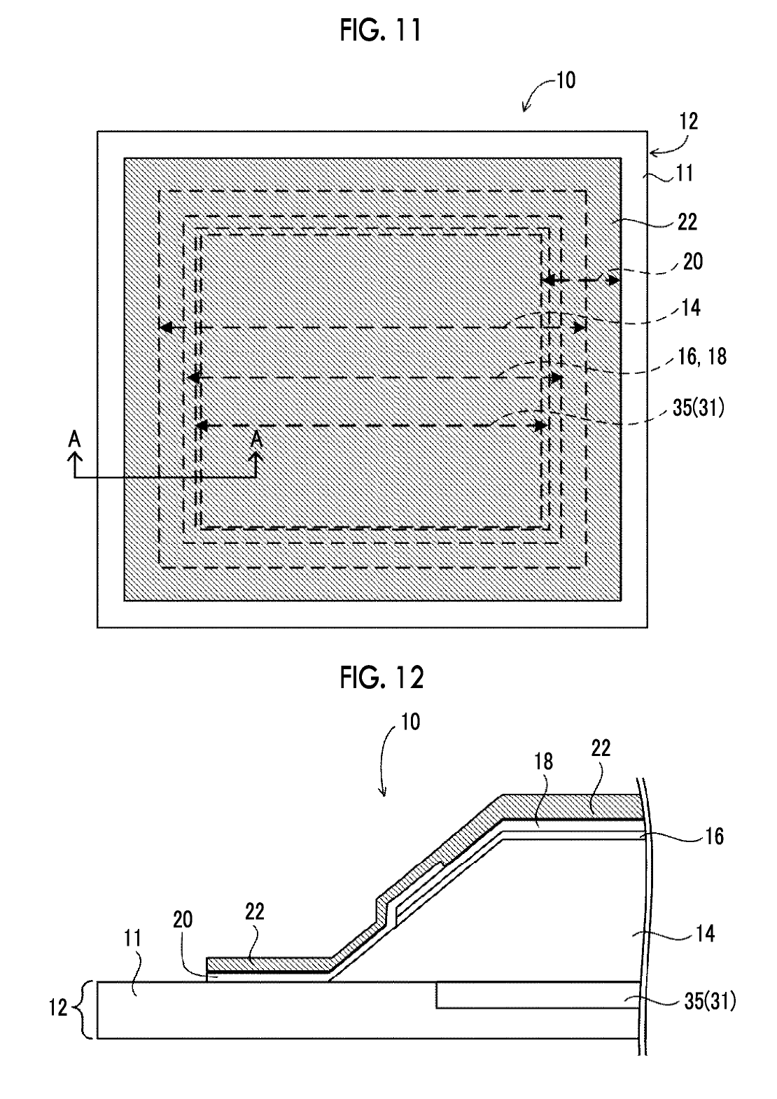

FIG. 11 is a plan view of the radiation detector 10 of the present embodiment as seen from the side on which the conversion layer 14 is formed. Additionally, FIG. 12 is a cross-sectional view taken along line A-A of the radiation detector 10 in FIG. 11.

As illustrated in FIGS. 11 and 12, in the radiation detector 10 of the present embodiment, the adhesive layer 20 is provided on a region ranging from the TFT substrate 12 in the vicinity of the peripheral edge part of the conversion layer 14 to the outer peripheral part of the reflective layer 18 (pressure sensitive adhesive layer 16). That is, in the radiation detector 10 of the present embodiment, the adhesive layer 20 does not cover the entire upper surfaces of the reflective layer 18 and the conversion layer 14.

An example of a method of manufacturing the radiation detector 10 of the present embodiment illustrated in FIGS. 11 and 12 includes the following method.

The sheet (film) of the pressure sensitive adhesive layer 16 having the desired size tailored for the radiation detector 10 and the sheet of the reflective layer 18 having the desired size are bonded together and prepared in advance.

Meanwhile, the conversion layer 14 is formed directly on the TFT substrate 12, by the vapor-phase deposition methods as described above.

Thereafter, the bonded pressure sensitive adhesive layer 16 and the reflective layer 18 that have been prepared in advance are bonded to the TFT substrate 12 on which the conversion layer 14 is formed.

Moreover, the adhesive layer 20 is provided at the peripheral edge part of the sheet of the protective layer 22 having the desired size. A method of providing the adhesive layer 20 is a method depending on the material of the adhesive layer 20, similarly to that described above in the first embodiment. For example, the adhesive layer 20 may be provided by application or bonding.

Thereafter, the sheet of the protective layer 22 on which the adhesive layer 20 is provided is bonded to the TFT substrate 12 on which the conversion layer 14, the pressure sensitive adhesive layer 16, and the reflective layer 18 are provided in order to bring about a state where the outer peripheral part that is the end part of the reflective layer 18 is covered with the adhesive layer 20.

In addition, in a case where the above bonding is performed, particularly in a case where the protective layer 22 in which the adhesive layer 20 is provided is bonded onto the reflective layer 18, it is preferable to perform the bonding under reduced pressure in order to suppress that air or the like enters between the portion of the protective layer 22 on which the adhesive layer 20 is not provided, and the reflective layer 18.

In this way, according to the radiation detector 10 of the present embodiment illustrated in FIGS. 11 and 12, the adhesive layer 20 covers the outer peripheral part that is the end part of the reflective layer 18 and covers the side surface of the reflective layer 18. Therefore, the peeling of particularly the end part of the reflective layer 18 is suppressed by the adhesive layer 20.

Additionally, according to the radiation detector 10 of the present embodiment, the adhesive layer 20 does not cover the entire upper surface of the conversion layer 14. Therefore, it is possible to suppress that radiation is attenuated by being transmitted through the adhesive layer 20 until the radiation is radiated from the protective layer 22 side and reaches the conversion layer 14.

### Fifth Embodiment

Next, a fifth embodiment will be described. In addition, the radiation detector 10 of the present embodiment is different from the third embodiment in terms of the region where the pressure sensitive adhesive layer 16 and the reflective layer 18 are provided, and is substantially the same as that of the fourth embodiment in terms of the region where the adhesive layer 20 is provided. The regions where the pressure sensitive adhesive layer 16, the reflective layer 18, and the adhesive layer 20 are provided will be described with reference to the drawings.

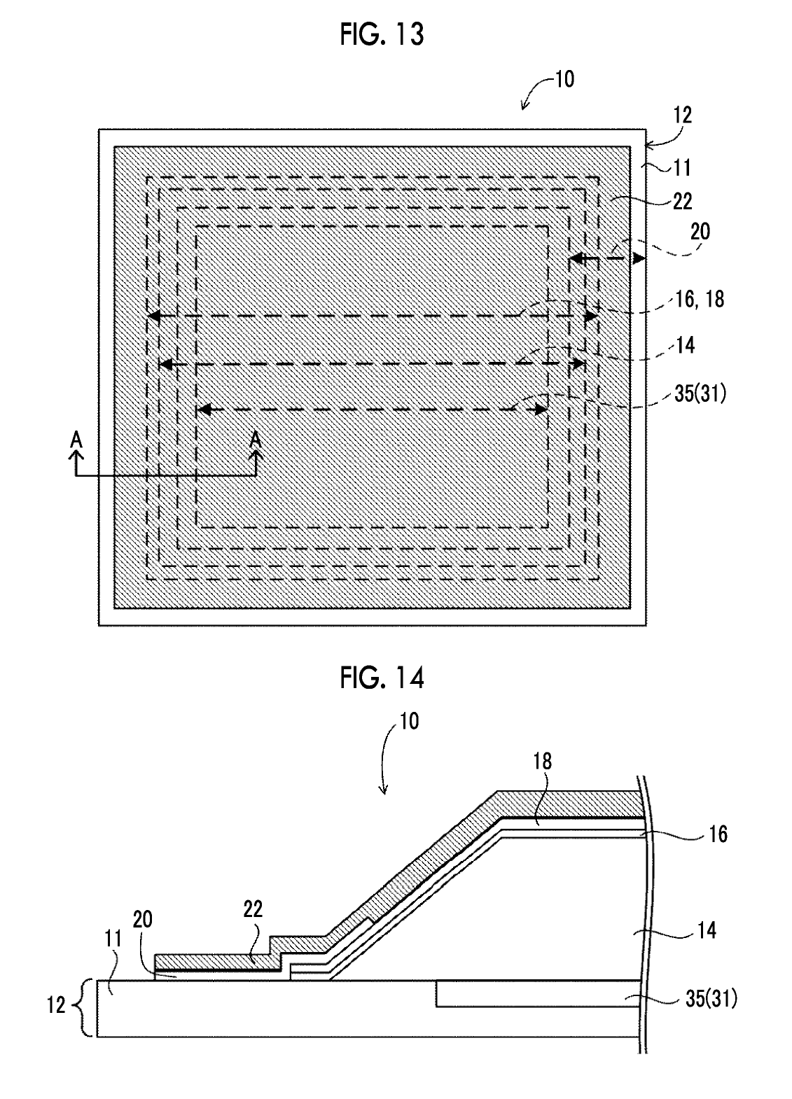

FIG. 13 is a plan view of the radiation detector 10 of the present embodiment as seen from the side on which the conversion layer 14 is formed. Additionally, FIG. 14 is a cross-sectional view taken along line A-A of the radiation detector 10 in FIG. 13.

As illustrated in FIGS. 13 and 14, in the radiation detector 10 of the present embodiment, the pressure sensitive adhesive layer 16 and the reflective layer 18 are provided in the entire region on the conversion layer 14 including the central part and the peripheral edge part, and a region on the TFT substrate 12 in the vicinity of the outer periphery of the conversion layer 14. Additionally, in the radiation detector 10 of the present embodiment, the adhesive layer 20 is provided on the region ranging from the TFT substrate 12 in the vicinity of the peripheral edge part of the conversion layer 14 to the outer peripheral part of the reflective layer 18 (pressure sensitive adhesive layer 16).

In addition, the radiation detector 10 of the present embodiment can be manufactured, for example, by the same manufacturing method as the manufacturing method of the radiation detector 10 that has been described above in the fourth embodiment.

In this way, according to the radiation detector 10 of the present embodiment illustrated in FIGS. 13 and 14, similarly to the radiation detector 10 of the fourth embodiment, the adhesive layer 20 covers the outer peripheral part and the side surface of the reflective layer 18. Therefore, the peeling of particularly the end part of the reflective layer 18 is suppressed by the adhesive layer 20.

Additionally, accordingly, the radiation detector 10 of the present embodiment, the pressure sensitive adhesive layer 16 and the reflective layer 18 reach the TFT substrate 12. Therefore, the reflective layer 18 can be more stably fixed to the TFT substrate 12 and the conversion layer 14.

Additionally, according to the radiation detector 10 of the present embodiment, the reflective layer 18 covers the entire upper surface of the conversion layer 14. Therefore, the light from the conversion layer 14 is easily reflected. Moreover, according to the radiation detector 10 of the present embodiment, the adhesive layer 20 does not cover the entire upper surface of the conversion layer 14. Therefore, it is possible to suppress that radiation is attenuated by being transmitted through the adhesive layer 20 until the radiation is radiated from the protective layer 22 side and reaches the conversion layer 14.

### Sixth Embodiment

In the above respective embodiments, the radiation detector 10 comprising the TFT substrate 12 in which the pixel region 35 of the base material 11 is provided with the pixel array 31. In the present embodiment, the radiation detector 10 having no base material 11 will be described.

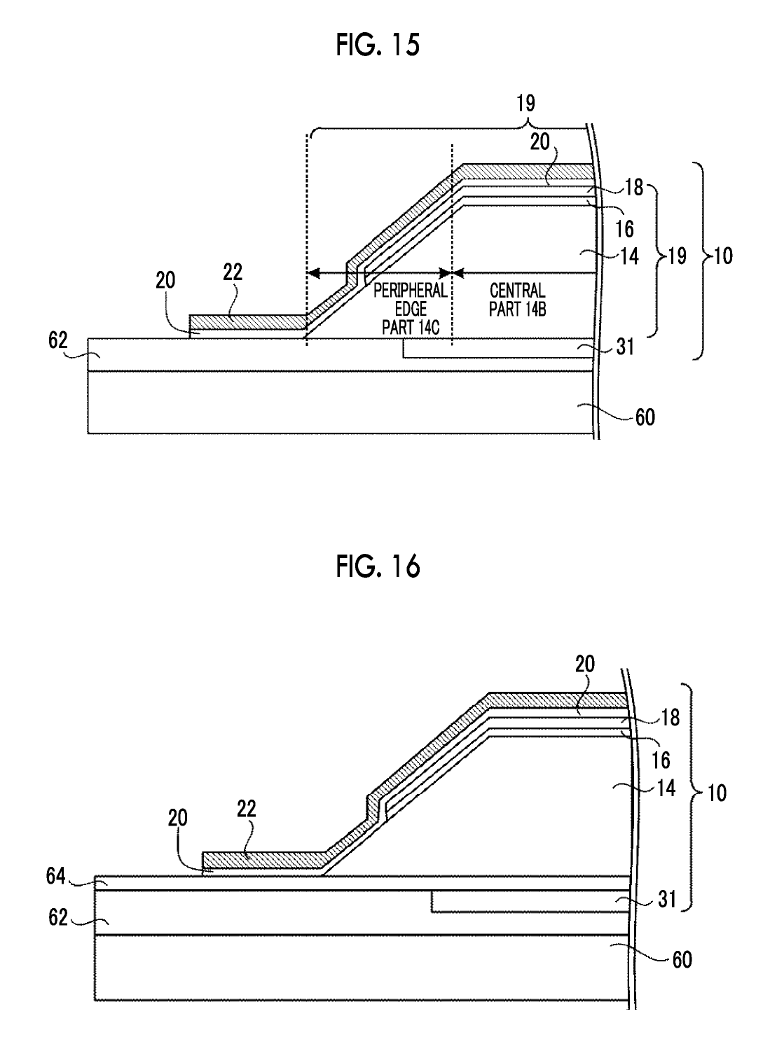

A cross-sectional view of an example of the radiation detector 10 of the present embodiment is illustrated in FIG. 15. As illustrated in FIG. 15, the radiation detector 10 of the present embodiment comprises the pixel array 31, the conversion layer 14, the pressure sensitive adhesive layer 16, the reflective layer 18, the adhesive layer 20, and the protective layer 22, similarly to the radiation detector 10 (refer to FIGS. 2 and 3) of the first embodiment.

Additionally, as illustrated in FIG. 15, the radiation detector 10 of the present embodiment is different from the radiation detector 10 (refer to FIGS. 3 and 4) of the first embodiment in that the radiation detector 10 of the present embodiment does not comprise the base material 11 (TFT substrate 12).

The radiation detector 10 of the present embodiment is manufactured by providing the pixel array 31 on a substrate 60 to be a support via a peeling layer 62, then further forming the conversion layer 14, the pressure sensitive adhesive layer 16, the reflective layer 18, the adhesive layer 20, and the protective layer 22, and then, peeling the substrate 60 by the peeling layer 62. Since the radiation detector 10 of the present embodiment is manufactured in this way, the radiation detector 10 does not comprise the base materials 11, such as the glass substrate.

As the substrate 60, for example, the glass substrate or the like can be used similarly to the support 50 described with reference to the above FIG. 6. Additionally, the peeling layer 62 is one for peeling the substrate 60 from the pixel array 31, and is formed of an organic or inorganic material to be activated by at least one method of a thermal method, an optical method, or a chemical method. A specific example of the method of activating the peeling layer 62 includes the etching.

Additionally, as in an example illustrated in FIG. 16, the radiation detector 10 of the present embodiment may comprise another layer having a predetermined function, such as a barrier layer 64, between the pixel array 31 and the conversion layer 14. In the radiation detector 10 illustrated in FIG. 16, by providing the barrier layer 64, it is possible to suppress that the pixels 30 deteriorate as a fluorescent material included in the conversion layer 14 diffuses in the pixel array 31. Additionally, the barrier layer 64 functions as an etching stop in a case where the peeling layer 62 is activated by the etching. As such a barrier layer 64, an organic material, such as silicon nitride, or an inorganic material, such as polyimide or benzocyclobutene (BCB), can be applied.

In this case, the adhesive layer 20 is directly fixed (adhered) to a portion of surface of the barrier layer 64. Accordingly, according to the radiation detector 10 illustrated in FIG. 16, the peeling of the reflective layer 18, particularly the peeling of the end part of the reflective layer 18 is suppressed.

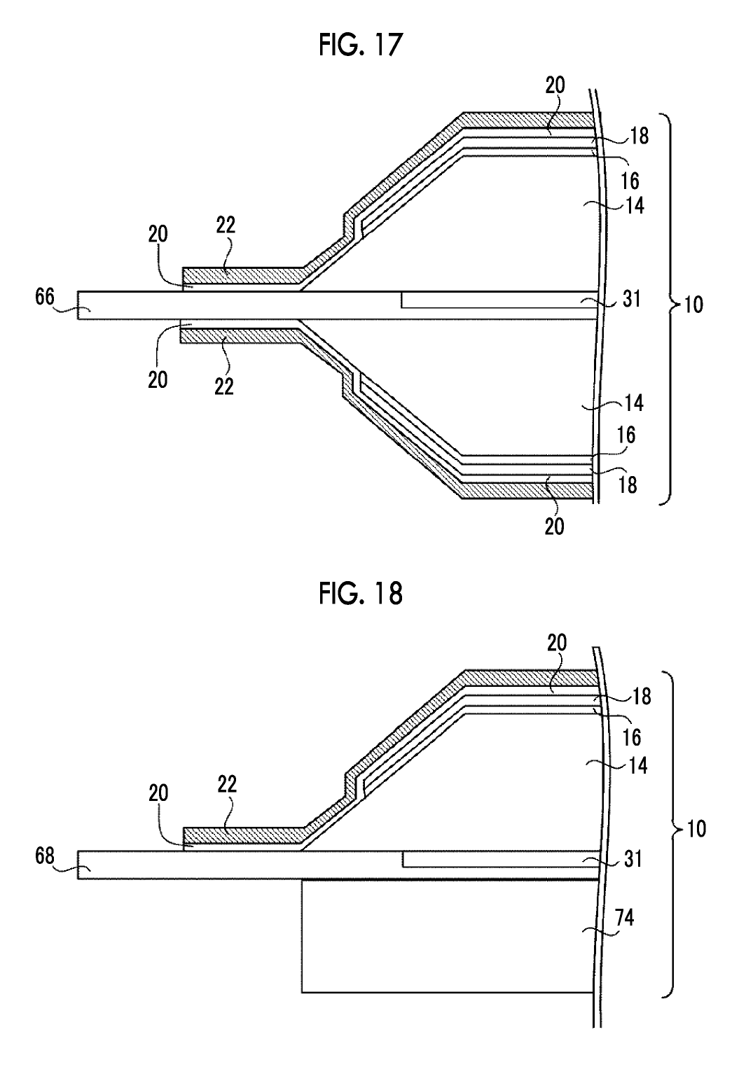

Additionally, the radiation detector 10 of the present embodiment may further comprise a conversion layer that faces the conversion layer 14 across the pixel array 31. For example, in an example illustrated in FIG. 17, a state where, across the pixel array 31 and a barrier layer 66, a pair of the conversion layers 14 faces each other, a pair of the pressure sensitive adhesive layers 16 faces each other, a pair of the reflective layers 18 faces each other, a pair of the adhesive layers 20 faces each other, and a pair of the protective layers 22 faces each other is illustrated. As the barrier layer 66 in this case, the same one as the above barrier layer 64 can be used.

Additionally, for example, in an example illustrated in FIG. 18, a state where a conversion layer 74 is further provided to face the conversion layer 14 across the pixel array 31 and a pressure sensitive adhesive layer 68 is illustrated. As the pressure sensitive adhesive layer 68 in this case, the same one as the above pressure sensitive adhesive layer 16 can be used. Additionally, as the conversion layer 74 in this case, one in which GOS (Gd₂O₂S:Tb) or the like is dispersed in a binder, such as resin, can be applied.

Additionally, in the radiation detector 10 of the present embodiment, for example in a case where the radiation detector 10 comprises the two conversion layers that face each other as in the radiation detector 10 illustrated in FIG. 17 or the radiation detector 10 illustrated in FIG. 18, pixel arrays 31 corresponding to the respective conversion layers may be provided between the conversion layers. In other words, the radiation detector 10 may comprise two sets of pixel arrays 31 and conversion layers (the conversion layer 14 or the conversion layer 74) in a state where the surfaces to be irradiated with radiation face each other. In addition, in this case, a shielding layer may be provided between the conversion layers in order to reduce the optical crosstalk between the pixel arrays 31.

In this way, in the radiation detector 10 of the present embodiment illustrated in FIGS. 15 to 18, the adhesive layer 20 and the protective layer 22 cover the entire (the entire upper surface and the side surface) reflective layer 18. Additionally, the adhesive layer 20 and the protective layer 22 are fixed to the substrate 60.

Accordingly, according to the radiation detector 10 of the present embodiment, the peeling of the reflective layer 18 with respect to the conversion layer 14, particularly the peeling of the end part of the reflective layer 18 with respect to the conversion layer 14 is suppressed. In addition, in the present embodiment, the end part of the reflective layer 18 means the region within the predetermined range in the vicinity of the outer periphery of the reflective layer 18.

As described above, the radiation detectors 10 of the above embodiments have a portion in which the TFT substrate 12 or the substrate 60, the pixel array 31 in which the plurality of pixels 30 that accumulates the electric charges generated in accordance with the light converted from radiation is formed, the conversion layer 14 that converts the radiation into light, the light-transmissive pressure sensitive adhesive layer 16, the reflective layer 18 that reflects the light converted by the conversion layer 14, and the adhesive layer 20 that covers at least the region ranging from the end part of the reflective layer 18 to the surface of the substrate 60 or the TFT substrate 12 are provided in order. The pixel array 31 is provided in the pixel region 35 of the TFT substrate 12 or in the substrate 60 via the peeling layer 52.

In this way, according to the radiation detectors 10 of the above respective embodiments, the entirety or the outer peripheral part of the reflective layer 18 is covered with the adhesive layer 20 having an adhesive force stronger than the adhesive force of the pressure sensitive adhesive layer 16. Therefore, the end part of the reflective layer 18 can be more firmly fixed.

Therefore, according to the radiation detectors 10 of the above respective embodiments, the peeling of the end part of the reflective layer 18 can be suppressed.

By suppressing the peeling of the end part of the reflective layer 18, not only the end part but also the peeling of the entire reflective layer 18 is suppressed.

In a case where the reflective layer 18 is peeled, light is no light is reflected toward the TFT substrate 12 side. Therefore, there is a concern that the sensitivity of the radiation detector 10 decreases. In contrast, by suppressing the peeling of the reflective layer 18, the decrease in sensitivity can be suppressed, the deterioration of the image quality of a radiographic image to be obtained by the radiation detector 10 can be suppressed.

Additionally, in a case where the reflective layer 18 is peeled, moisture, such as humidity, easily enters the inside of the radiation detector 10 from the peeled portion. The conversion layer 14, particularly the conversion layer 14 using CsI is weak to moisture. Therefore, in a case where the moisture enters the inside of the radiation detector 10, there is a concern to that the image quality of a radiographic image to be obtained by the radiation detector 10 may deteriorate. In contrast, by suppressing the peeling of the reflective layer 18, the entry of the moisture can be suppressed, the deterioration of the image quality of a radiographic image to be obtained by the radiation detector 10 can be suppressed.

Additionally, in a case where the side surface of the reflective layer 18 is exposed, there is a concern that moisture, such as humidity, enters the inside of the radiation detector 10 from the exposed spot. However, in the radiation detectors 10 of the above respective embodiments, the side surface of the reflective layer 18 is covered with the adhesive layer 20 and the protective layer 22. Therefore, a moisture preventing effect can be enhanced.

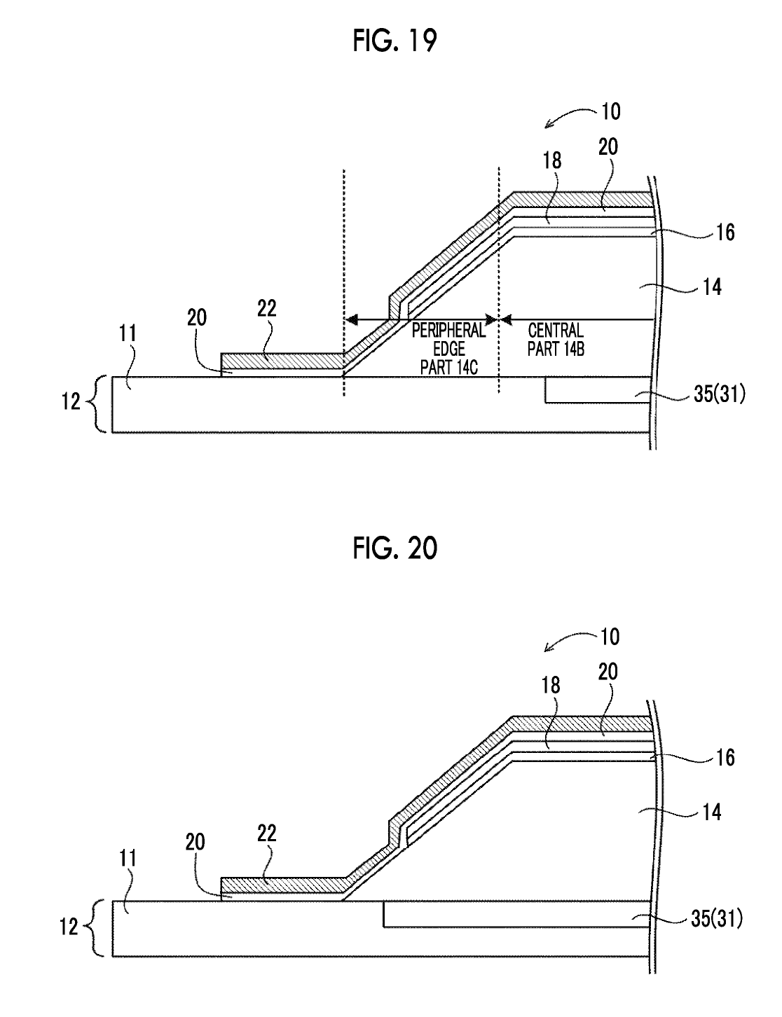

In addition, the size of the pixel array 31 (pixel region 35) is not limited to that of the above respective embodiments. For example, in the above respective embodiments, a form has been described in which the size of the pixel array 31 (pixel region 35) is larger than the size of the central part 14B of the conversion layer 14, and the outer periphery of the pixel array 31 (pixel region 35) reaches the peripheral edge part 14C of the conversion layer 14. However, as in an example illustrated in FIG. 19, a form may be adopted in which the size of the pixel array 31 (pixel region 35) is smaller than the size of the central part 14B of the conversion layer 14, and the outer periphery of the pixel array 31 (pixel region 35) is within the central part 14B. The quantity of light to be converted from radiation in the conversion layer 14 tends to decrease in a case where the thickness of the conversion layer 14 decreases. However, in the four of the example illustrated in FIG. 19, the thickness of the conversion layer 14 on the pixel array 31 (pixel region 35) becomes substantially uniform. Therefore, the sensitivity characteristics of the pixel region 35 are improved.

Additionally, for example, the size of the pixel array 31 (pixel region 35) may be larger than that of the radiation detectors 10 of the above respective embodiments. For example, in the above respective embodiments, the size of the pixel array 31 (pixel region 35) is smaller than the size of the region where the reflective layer 18 is provided, and the pixel array 31 (pixel region 35) is covered with the reflective layer 18. However, as in an example illustrated in FIG. 20, a form may be adopted in which the pixel array 31 (pixel region 35) is larger than the reflective layer 18, and there is a region of the pixel array 31 (pixel region 35) that is not covered with the reflective layer 18. In addition, in the form in which the reflective layer 18 covers the entire pixel array 31 (pixel region 35) as in the radiation detectors 10 of the above respective embodiments, the image quality of a radiographic image to be obtained depending on the pixels 30 located at the outer peripheral part of the pixel array 31 (pixel region 35) is improved compared to the radiation detector 10 illustrated in FIG. 20.

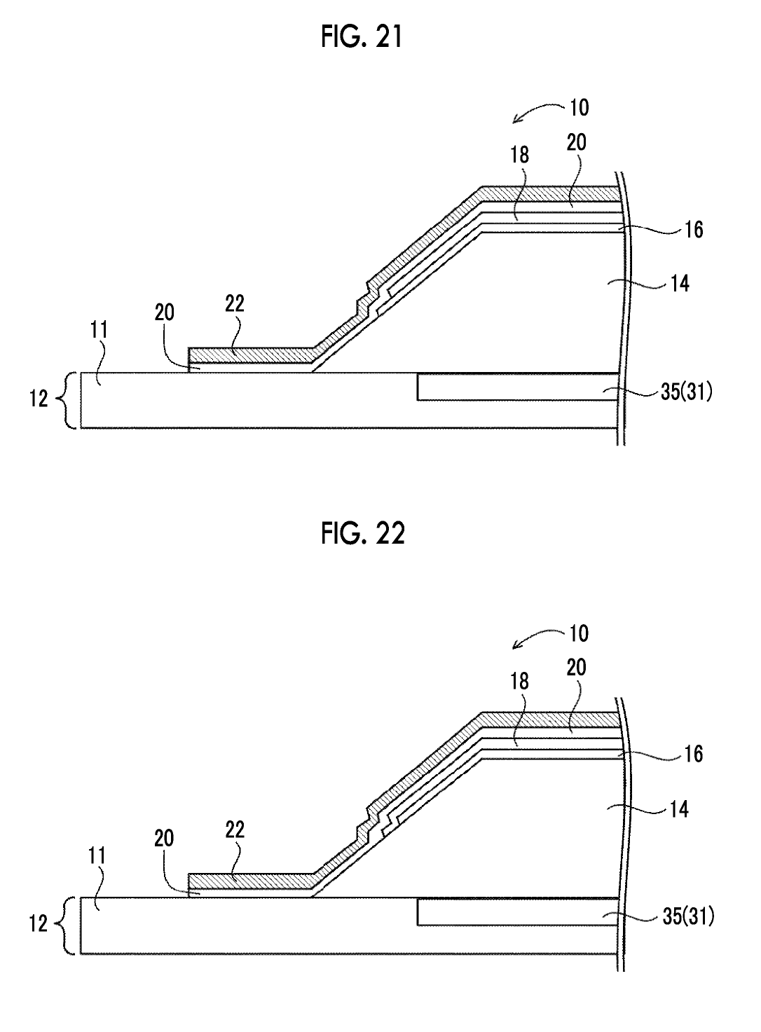

Additionally, in the radiation detectors 10 of the above respective embodiments, a form in which the region where the reflective layer 18 is provided and the region where the pressure sensitive adhesive layer 16 is provided are the same (are the same in size) has been described. However, the invention is not limited to this aspect. For example, as in an example illustrated in FIG. 21, a form may be adopted in which the reflective layer 18 is provided in a partial region of the upper surface of the pressure sensitive adhesive layer 16 instead of the entire upper surface of the pressure sensitive adhesive layer 16. Additionally, as in an example illustrated in FIG. 22, a form may be adopted in which the region where the reflective layer 18 covers the pressure sensitive adhesive layer 16 such that the reflective layer 18 is provided is wider than the region where the pressure sensitive adhesive layer 16 (the reflective layer 18 is larger). In the radiation detector 10 of any of the forms in FIGS. 21 and 22, the peeling of particularly the end part of the reflective layer 18 is suppressed by the adhesive layer 20 by covering the outer peripheral part that is an end part of the reflective layer 18 by the adhesive layer 20. In addition, as in the radiation detectors 10 of the above respective embodiments and the radiation detector 10 illustrated as an example in FIG. 21, the peeling of the reflective layer 18 is further suppressed in a case where the pressure sensitive adhesive layer 16 is provided at the entire lower part of the reflective layer 18.

Additionally, in the above respective embodiments, as illustrated in FIG. 1, an aspect in which the pixels 30 are two-dimensionally arranged in a matrix has been described. However, the invention is not limited to the aspect, and the pixels 30 may be one-dimensionally arranged or may be arranged in a honeycomb shape. Additionally, the shape of the pixels is also not limited, and may be a rectangular shape, or may be a polygonal shape, such as a hexagonal shape. Moreover, it goes without saying that the shape of the pixel array 31 (pixel region 35) is also not limited.

Additionally, the shape or the like of the conversion layer 14 is not limited to the above respective embodiments. In the above respective embodiments, an aspect in which the shape of the conversion layer 14 is a rectangular shape like the shape of the pixel array 31 (pixel region 35) has been described. However, the shape of the conversion layer 14 may not be the same shape as the pixel array 31 (pixel region 35). Additionally, the shape of the pixel array 31 (pixel region 35) may not be a rectangular shape, but may be, for example, other polygonal shapes or a circular shape.

In addition, in the above respective embodiments, as an example, a form in which the conversion layer 14 of the radiation detector 10 is the scintillator including CsI has been described. However, the conversion layer 14 may be a scintillator in which GOS or the like is dispersed in a binder, such as resin. The conversion layer 14 using GOS is formed, for example, by directly applying the binder having the GOS dispersed therein onto the TFT substrate 12, the peeling layer 62, and the like and then drying and solidifying the binder. As a method of forming the conversion layer 14, for example, a Giza method of applying an application liquid to a region where the conversion layer 14 is formed while controlling the thickness of an applied film may be adopted. In addition, in this case, surface treatment for activating the surface of the pixel array 31 may be performed before the binder having the GOS dispersed therein is applied. Additionally, an interlayer insulation film may be provided as a surface protective film on the surface of the pixel array 31.

In a case where the conversion layer 14 using GOS is directly applied onto the surface of the pixel array 31, the end part sags, and similarly to the above-described conversion layer 14 using the CsI, the inclined part is formed at the end part of the conversion layer 14, and the end part of the reflective layer 18 is easily peeled.

In contrast, as described above, the entirety or the outer peripheral part of the reflective layer 18 is covered with the adhesive layer 20 having an adhesive force stronger than the adhesive force of the pressure sensitive adhesive layer 16. Accordingly, the end part of the reflective layer 18 can be more firmly fixed. Therefore, also in the radiation detector 10 having the conversion layer 14 using GOS, the peeling of the end part of the reflective layer 18 can be suppressed.

In addition, the radiation detectors 10 of the above respective embodiments may be applied to an irradiation side sampling (ISS) type radiographic imaging apparatus in which radiation is radiated from the TFT substrate 12 side, or may be applied to a penetration side sampling (PSS) type radiographic imaging apparatus in which radiation is radiated from the conversion layer 14 side.

A cross-sectional view of an example in a state where the radiation detector 10 of the first embodiment is applied to an irradiation side sampling type radiographic imaging apparatus 1 is illustrated in FIG. 23.

As illustrated in FIG. 23, the radiation detector 10, the power source unit 108, and a control board 110 are provided side by side in a direction intersecting an incidence direction of radiation within the housing 120. The radiation detector 10 is provided in a state where a side where the conversion layer 14 of the pixel array 31 is not provided faces an imaging surface 120A side of the housing 120 that is irradiated with radiation transmitted through the subject.

The control board 110 is a board in which an image memory 210 for storing image data according to the electric charges read from the pixels 30 of the pixel array 31, a control unit 212 for controlling reading or the like of the electric charges from the pixels 30, and the like are formed, and is electrically connected to the pixels 30 of the pixel array 31 by a flexible cable 112 including a plurality of signal wiring lines. In addition, in the radiographic imaging apparatus 1 illustrated in FIG. 23, the control board 110 is a so-called chip on film (COF) in which a drive unit 103 for controlling the switching states of the TFTs 32 of the pixels 30 under the control of the control unit 212, and a signal processing unit 104 for creating and outputting image data according to the electric charges read from the pixels 30 are provided on the flexible cable 112. However, at least one of the drive unit 103 or the signal processing unit 104 may be formed in the control board 110.

Additionally, the control board 110 is connected to the power source unit 108, which supplies electrical power to the image memory 210, the control unit 212, and the like that are formed in the control board 110, by a power source line 114.

A sheet 116 is further provided on a side from which the radiation transmitted through the radiation detector 10 is emitted, within the housing 120 of the radiographic imaging apparatus 1 illustrated in FIG. 23. The sheet 116 is, for example, a copper sheet. The copper sheet does not easily generate secondary radiation due to incident radiation, and therefore, has a function of preventing scattering to the rear side, that is, the conversion layer 14 side. In addition, it is preferable that the sheet 116 covers at least an entire surface of the conversion layer 14 from which radiation is emitted, and covers the entire conversion layer 14.

A protective layer 117 is further provided on a side (imaging surface 120A side) to which radiation is incident, within the housing 120 of the radiographic imaging apparatus 1 illustrated in FIG. 23. As the protective layer 117, moistureproof films, such as an ALPET (registered trademark) sheet obtained by laminating aluminum, for example by adhering aluminum foil, to the insulating sheet (film), a parylene (registered trademark) film, and an insulating sheet (film), such as polyethylene terephthalate, can be applied. The protective layer 117 has a moistureproof function and an antistatic function with respect to the pixel array 31. For that reason, it is preferable that the protective layer 117 covers at least the entire surface of the pixel array 31 on the side to which radiation is incident, and it is preferable to cover the entire surface of the TFT substrate 12 on the side to which radiation is incident.

In addition, although FIG. 23 illustrates a form in which both the power source unit 108 and the control board 110 are provided on one side of the radiation detector 10, specifically, on one side of a rectangular pixel array 31, a position where the power source unit 108 and the control board 110 are provided is not limited to the form illustrated in FIG. 23. For example, the power source unit 108 and the control board 110 may be provided so as to be respectively decentralized onto two facing sides of the pixel array 31, or may be provided so as to be respectively decentralized onto two adjacent sides.

Additionally, a cross-sectional view of another example in a state where the radiation detector 10 of the first embodiment is applied to the irradiation side sampling type radiographic imaging apparatus 1 is illustrated in FIG. 23.

As illustrated in FIG. 23, the power source unit 108 and the control board 110 are provided side by side in the direction intersecting the incidence direction of radiation within the housing 120, and the radiation detector 10, the power source unit 108, and the control board 110 are provided side by side in the incidence direction of radiation.

Additionally, in the radiographic imaging apparatus 1 illustrated in FIG. 23, a base 118 that supports the radiation detector 10 and the control board 110 is provided between the control board 110 and the power source unit 108, and the sheet 116. For example, carbon or the like is used for the base 118.

In addition, it goes without saying that the configurations, manufacturing methods, and the like of the radiation detectors 10, and the like that are described in the above respective embodiments are merely examples, and can be modified in accordance with situations without departing from the scope of the invention.

The disclosure of Japanese Patent Application No. 2016-251803, filed on Dec. 26, 2016, and the disclosure of Japanese Patent Application No. 2017-126683, filed on Jun. 28, 2017 are incorporated into the present specification by reference in its entirety.

All documents, patent applications, and technical standards described in the present specification are incorporated in the present specification by reference in their entireties to the same extent as in a case where the individual documents, patent applications, and technical standards are specifically and individually written to be incorporated by reference.

What is claimed is:

1. A radiation detector comprising a portion in which
a substrate;
a pixel array that is provided in a pixel region of the substrate and that has a plurality of pixels for accumulating electric charges generated in accordance with light converted from radiation;
a conversion layer that converts the radiation into light;
a light-transmissive pressure sensitive adhesive layer;
a reflective layer that reflects the light converted by the conversion layer; and
an adhesive layer that covers at least a region ranging from an end part of the reflective layer to a surface of the substrate
are provided in order.

2. The radiation detector according to claim 1, wherein the conversion layer covers a region of the substrate where the pixel array is provided.

3. The radiation detector according to claim 1, wherein the pressure sensitive adhesive layer covers a region including a central part of the conversion layer.

4. The radiation detector according to claim 1, wherein the pressure sensitive adhesive layer covers the conversion layer in a region enclosing the pixel array.

5. The radiation detector according to claim 1, wherein:
a peripheral edge part of the conversion layer has an inclination such that a thickness thereof decreases toward an outside, and
an outer peripheral part of the reflective layer is located at the peripheral edge part of the conversion layer.

6. The radiation detector according to claim 1, wherein:
a peripheral edge part of the conversion layer has two different heights, and
an outer peripheral part of the reflective layer is located at the peripheral edge part of the conversion layer.

7. The radiation detector according to claim 1, wherein the adhesive layer covers the reflective layer.

8. The radiation detector according to claim 1, wherein the adhesive layer covers the conversion layer.

9. The radiation detector according to claim 1, wherein the pressure sensitive adhesive layer is provided between the reflective layer and the conversion layer.

10. The radiation detector according to claim 1, wherein a material of the reflective layer is white PET.

11. The radiation detector according to claim 10, wherein a thickness of the reflective layer is 10 μM or more and 40 μm or less.

12. The radiation detector according to claim 1, wherein the reflective layer is provided in a region corresponding to the pixel array.

13. The radiation detector according to claim 1, wherein a thickness of the pressure sensitive adhesive layer is 2 μm or more and 7 μm or less.

14. The radiation detector according to claim 1, wherein the conversion layer includes columnar crystals of CsI.

15. The radiation detector according to claim 1, wherein the conversion layer is a resin layer in which GOS applied to the pixel array is dispersed.

16. The radiation detector according to claim 1, further comprising a protective layer that covers a laminate including the conversion layer, the pressure sensitive adhesive layer laminated on the conversion layer, and the reflective layer laminated on the pressure sensitive adhesive layer, and the adhesive layer.

17. The radiation detector according to claim 1, wherein an inclination angle of at least a portion of the peripheral edge part of the conversion layer is 90° or more and 166° or less.

18. A radiographic imaging apparatus comprising:
the radiation detector according to claim 1;
a control unit that outputs control signals for reading electric charges accumulated in the plurality of pixels;
a drive unit that allows the electric charges to be read from the plurality of pixels in accordance with the control signals; and
a signal processing unit to which electrical signals according to the electric charges read from the plurality of pixels are input, and which generates image data according to the input electrical signals to output the image data to the control unit.

19. A radiation detector comprising a portion in which
a substrate;
a pixel array that is provided on the substrate via a peeling layer and that has a plurality of pixels for accumulating electric charges generated in accordance with light converted from radiation;
a conversion layer that converts the radiation into light;
a light-transmissive pressure sensitive adhesive layer;
a reflective layer that reflects the light converted by the conversion layer; and
an adhesive layer that covers at least a region ranging from an end part of the reflective layer to a surface of the substrate
are provided in order.

20. The radiation detector according to claim 19, wherein the conversion layer covers a region of the substrate where the pixel array is provided.

21. A radiation detector comprising a portion in which
a barrier layer;
a pixel array that has a plurality of pixels for accumulating electric charges generated in accordance with light converted from radiation;
a conversion layer that converts the radiation into light;
a light-transmissive pressure sensitive adhesive layer;
a reflective layer that reflects the light converted by the conversion layer; and
an adhesive layer that covers at least a region ranging from an end part of the reflective layer to a surface of the barrier layer
are provided in order.

22. The radiation detector according to claim 21, wherein the conversion layer covers a region of the barrier layer where the pixel array is provided.
